(19) United States
(12) Patent Application Publication
(10) Pub. No.: US 2023/0000389 A1
(43) Pub. Date: Jan. 5, 2023

(54) MEASUREMENT OF ALVEOLAR DEAD SPACE USING SEQUENTIAL GAS DELIVERY

(71) Applicant: THORNHILL SCIENTIFIC INC., North York (CA)

(72) Inventors: Roy KATZNELSON, Toronto (CA); Rafay Ali KHAN, Markham (CA); Olivia SOBCZYK, Etobicoke (CA); James DUFFIN, Toronto (CA); Joseph Arnold FISHER, Thornhill (CA)

(21) Appl. No.: 17/780,816

(22) PCT Filed: Nov. 30, 2020
(86) PCT No.: PCT/IB2020/061281

§ 371 Date: May 27, 2022

Related U.S. Application Data
(60) Provisional application No. 62/941,837, filed on Nov. 28, 2019.

(51) Int. Cl. A61B 5/091; A61M 16/12

(52) U.S. Cl. CPC A61B 5/091; A61M 16/12; A61M 2016/0027

(57) ABSTRACT

Alveolar dead space of a subject is determined by measuring an end tidal partial pressure of carbon dioxide during a sequence of normal breaths of the subject and, during a sequence of deep breaths by the subject, delivering a first volume of a first gas to the subject over a first portion of each inspiration by the subject. The first volume is less than or equal to an expected alveolar volume of the subject when the subject is breathing normally. A second volume of a second gas is delivered to the subject over a second portion of each inspiration. The second gas includes a neutral gas. An end tidal partial pressure of carbon dioxide is measured during the sequence of deep breaths. The alveolar dead space is computed using the end tidal partial pressures of carbon dioxide measured during the sequence of normal breaths and the sequence of deep breaths.

### CROSS-REFERENCE TO RELATED APPLICATION

This application claims priority to U.S. provisional app. Ser. No. 62/941,837, filed Nov. 28, 2019, and incorporated herein by reference.

### BACKGROUND

The mammalian lung includes two functional components: 1) airways for convection of gases to and from 2) the sac-like alveoli. The alveolar walls are surrounded by a mesh of tiny blood vessels called capillaries. Gases such as oxygen and carbon dioxide diffuse between the gas in the alveoli and blood in the capillaries.

The airways are composed of a set of branching thick walled tubes that do not engage in gas exchange with the blood, generally termed “dead space.” Airway dead space is specifically referred to as the “anatomical dead space.”

Some of the alveoli also do not participate in gas exchange. This may be due to their walls being too thickened to readily allow diffusion, their capillaries having reduced or absent perfusion of blood, or their lacking capillaries altogether. This is called “alveolar dead space.”

### SUMMARY

According to one aspect of the present disclosure, a method of determining alveolar dead space of a subject includes measuring an end tidal partial pressure of carbon dioxide during a sequence of normal breaths of the subject and, during a sequence of deep breaths by the subject, delivering a first volume of a first gas to the subject over a first portion of each inspiration by the subject. The first volume is less than or equal to an expected alveolar volume of the subject when the subject is breathing normally. The method further includes delivering a second volume of a second gas to the subject over a second portion of each inspiration. The second gas includes a neutral gas. The method further includes measuring an end tidal partial pressure of carbon dioxide during the sequence of deep breaths and computing the alveolar dead space using the end tidal partial pressure of carbon dioxide measured during the sequence of normal breaths and the end tidal partial pressure of carbon dioxide measured during the sequence of deep breaths.

The method may further include computing the alveolar dead space based on a ratio of the end tidal partial pressure of carbon dioxide measured during the sequence of normal breaths to an alveolar partial pressure of carbon dioxide of the subject. The alveolar partial pressure of carbon dioxide of the subject is considered to be the end tidal partial pressure of carbon dioxide measured during the sequence of deep breaths.

The first gas may have a partial pressure of carbon dioxide that is less than a partial pressure of carbon dioxide of a gas exhaled by the subject.

The second gas may have a partial pressure of carbon dioxide corresponding to the partial pressure of carbon dioxide of the gas exhaled by the subject.

The first volume may be less than or equal to an expected alveolar volume of the subject when the subject is breathing normally.

According to another aspect of the present disclosure, the method may be used in an assessment of a medical condition selected from a group consisting of heart failure, congestive heart failure, adult respiratory distress syndrome, lung function after lung transplant, lung function after transplant, sepsis, trauma, and pulmonary and systemic disease.

According to another aspect of the present disclosure, a device for determining alveolar dead space of a subject includes a user interface device, a gas blender to receive source gases, and a processor connected to the user interface device and the gas blender to control the supply of gas to the subject. The processor is to control the gas blender to deliver a first gas and a second gas sequentially to the subject during a period of deep breathing by the subject. A volume of the first gas is about an expected alveolar volume of the subject when the subject is breathing normally. The second gas is a neutral gas. The first gas has a partial pressure of carbon dioxide that is less than that of the second gas. The processor is further to measure an end tidal partial pressure of carbon dioxide of the subject during the period of deep breathing, compute a representation of alveolar dead space using the end tidal partial pressure of carbon dioxide measured during deep breathing and an end tidal partial pressure of carbon dioxide of the subject obtained during normal breathing, and output the indication of the alveolar dead space at the user interface.

The processor may deliver the first gas and the second gas sequentially to the subject during a period of normal breathing and measure the end tidal partial pressure of carbon dioxide of the subject during the period of normal breathing.

The processor may compute the representation of alveolar dead space based on a ratio of the end tidal partial pressure of carbon dioxide of the subject obtained during normal breathing to an alveolar partial pressure of carbon dioxide of the subject. The alveolar partial pressure of carbon dioxide of the subject is considered to be the end tidal partial pressure of carbon dioxide measured during deep breathing.

The processor may compute the representation of alveolar dead space as a ratio of the alveolar dead space to a volume of gas entering the alveoli.

According to another aspect of the present disclosure, the device may be used to make an assessment of a medical condition selected from a group consisting of heart failure, congestive heart failure, adult respiratory distress syndrome, lung function after lung transplant, lung function after transplant, sepsis, trauma, and pulmonary and systemic disease.

### BRIEF DESCRIPTION OF THE DRAWINGS

FIG. 1A is a graph of a Bohr dead space.

FIG. 1B is a graph of an Enghoff dead space.

FIG. 2 is a graph of a Fowler dead space.

FIG. 3 is a block diagram of a device to measure an alveolar dead space of a subject.

FIG. 4 is a flowchart of a method of measuring an alveolar dead space of a subject.

FIG. 5A is a schematic diagram of gas distribution, equilibration, and mixing in relation to end tidal concentration of carbon dioxide in a two-region alveolar model under normal breathing.

FIG. 5B is a schematic diagram of gas distribution, equilibration, and mixing in relation to end tidal concentration of carbon dioxide in a two-region alveolar model under sequential gas delivery with normal or spontaneous breathing.

FIG. 6 is a schematic diagram of gas distribution, equilibration, and mixing in relation to equilibration of arterial carbon dioxide and carbon dioxide in exhaled gas at end of expiration in a two-region alveolar model under sequential gas delivery with deep breathing.

FIG. 7 is a plot of a measured ratio of alveolar dead space to volume of gas entering the alveoli plotted as partial pressure of carbon dioxide in exhaled gas at end of expiration versus time.

### DETAILED DESCRIPTION

Alveolar dead space (VDalv) is an important parameter in the assessment of lung diseases such as acute respiratory distress syndrome (ARDS), pulmonary embolization, chest trauma, prognosis in major surgery, and lung transplantation, as well as in the investigation of exercise tolerance and sport performance. It is also thought to be a major contributor to the proportional increased breathing in response to exertion in patients with heart failure.

It is difficult to measure precisely VDalv. However, it can be measured in combination with anatomical dead space (VDanat) as the “physiological dead space” (V_{Dphys}).

The volume of the physiological dead space (VDphys) can be calculated from how much it dilutes carbon dioxide (CO₂) in the perfused alveoli in a collection of exhaled gas. To do this, all the gas expired in, say, one minute is collected in a bag and then the fractional partial pressure of CO₂ (FCO₂) in the bag is measured by measuring the partial pressure of CO₂ (PCO₂) specific to the barometric pressure (P_{B}), where:

FCO₂=PCO₂/P_{B}

The concentration of CO₂ in the bag comes from all parts of the lung, so it is called the “mixed expired concentration” (FĒCO₂). The volume of the dead space (VD) can be measured or estimated based on comparing the mixed expired concentration (FĒCO₂) to the concentration of CO₂ in the inspired gas (FICO₂, 0%) and to the concentration of CO₂ in the alveoli (FACO₂), which is assumed to be the concentration measured from the end of a breath, i.e., the end tidal concentration of CO₂ (F_{ET}CO₂). Note that the end tidal partial pressure of carbon dioxide (P_{ET}CO₂) cannot be equal to the partial pressure of CO₂ in the alveoli (PACO₂) as it is a mixture of the PACO₂ and inspired gas coming out of the alveolar dead space (VDalv). However, as the PACO₂ is not otherwise measurable using conventional methodologies, this simplifying assumption is made. Another approximation of PACO₂ is the partial pressure of CO₂ in arterial blood (PaCO₂).

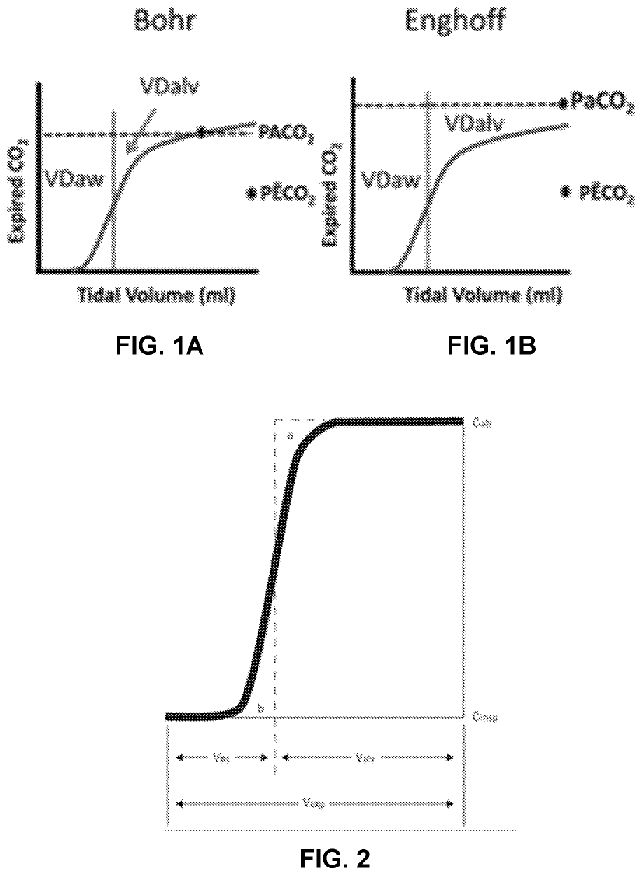

Two currently accepted ways of calculating physiological dead space (VDphys) are the Bohr method (FIG. 1A) and the Enghoff method (FIG. 1B). The subject inhales air and exhales into a container for multiple breaths. The volume of the bag is the sum of the volume of inspired gas or tidal volume (V_{T}). The partial pressure of carbon dioxide (PCO₂) in the bag is a mixture of PCO₂ from alveoli and all of the dead space, and is called mixed expired PCO₂ (PĒCO₂).

V_{T}FĒCO₂=(V_{T}−VDphys)×FACO₂ (Bohr dead space)

V_{T}×FĒCO₂=(V_{T}−VDphys)×FaCO₂ (Enghoff dead space)

For the Bohr dead space, the alveolar partial pressure of carbon dioxide (PACO₂) is assumed to be that corresponding to halfway along the expiratory plateau (Phase III). For the Enghoff dead space, PACO₂ is assumed to be equal to the partial pressure of CO₂ in arterial blood (PaCO₂). FIGS. 1A and 1B show that PaCO₂ is usually greater than the end tidal partial pressure of CO₂ (P_{ET}CO₂) resulting in the calculation of a larger physiological dead space (VDphys) (VDaw is the volume of the airways, or VDanat). In addition, the substitution of PaCO2 for PACO2 by Enghoff produces confusion in the interpretation of the mechanisms of dead space production. The substitution is only valid in an ideal lung, one with perfect ventilation to perfusion (\dot{V}/\dot{Q}) matching for all units, which cannot be expected in practice.

If physiological dead space (VDphys) can be determined, as above, then alveolar dead space (VDalv) can be computed as VDphys−VDanat, provided that anatomical dead space (VDanat) can also be determined.

As mentioned, anatomical dead space (VDanat) consists of the volume of the conducting airways, such as the trachea and bronchi. During inhalation, gas flows along the trachea, bronchi and eventually (mostly by diffusion) into the alveoli. The first gas inhaled during a breath reaches the alveoli. In the alveoli, it undergoes gas exchange with capillary blood to the point of equilibration so that the partial pressure of CO₂ and the partial pressure of O₂ of gas in the alveoli and capillary blood are the same. At the end of the expiration, the last approximately 150 ml of the breath remains in the anatomical dead space (VDanat). At the beginning of the next inspiration, the gas of the VDanat is the first to enter into the alveoli and joins with the gas in the functional residual capacity (FRC).

The traditional way to measure anatomical dead space (VDanat) is to take a breath of pure oxygen and exhale while measuring flow and nitrogen concentration. Nitrogen concentration rises when the VDanat flow passes the sensor measuring nitrogen concentration. This is the Fowler dead space.

FIG. 2 shows Fowler's illustration of the measurement of anatomical dead space, plotting exhaled concentration of nitrogen (C) following an inspired breath of 100% oxygen against exhaled volume (Vexp), where Cinsp represents the inspired nitrogen concentration (0%) and Calv represents the alveolar concentration of nitrogen. The vertical dashed line is positioned so that a and b subsume equal areas, and the intersection of the dashed line with the exhaled volume axis defines Vds, the anatomical dead space volume. Fowler noted that V_{Danat} was very close to 1 ml/lb in both men and women. With deep breaths, V_{Danat} may enlarge a little.

The Fowler method is but one example and other methods are suggested. However, many of such methods of measuring anatomical dead space (VDanat) have poor rationale and have not—indeed, cannot—be validated against any standard. Moreover, these measures become less accurate the more mixing there is of the alveolar gas with gas in the alveolar dead space (VDalv) and anatomical dead space (VDanat).

The conductive airway-alveolar interface constitutes the limit between the convective and diffusive transports of CO₂ within the lungs. As each terminal bronchial cluster has its own interface due to the asymmetry of airways, and due to mixing and diffusion of gas between that from the bronchi and that in the alveoli, there has to be a broad (as opposed to an abrupt) transition of the partial pressure of CO₂ (PCO₂) as measured at the sensor during an expiration. This gives rise to the slope in the transition from anatomical dead space gas to alveolar gas (also called Phase II slope). As a result, the airway-alveolar interface and thus the measure of the anatomical dead space (VDanat) is placed, merely by convention and for convenience (i.e., not by scientifically verified methods), at the midpoint of this Phase II. Engel and Paiva have provided theoretical evidence for this assumption, using mathematical simulation, yet it remains an assumption and not a measurement. The same extent of uncertainty applies to the calculation of alveolar dead space (VDalv). This lack of an accepted “gold standard” limits the ability to compare methodologies for anatomical and thus alveolar dead space measurement. Thus, confidence in measures reflect only the confidence in the measuring method.

Hence, while it is possible to measure physiological dead space (VDphys) and anatomical dead space (VDanat), as discussed above, so as to compute alveolar dead space (VDalv), this approach may be cumbersome, time-consuming, invasive, and inaccurate.

The present disclosure provides convenient, quick, non-invasive, and accurate techniques for the measurement of alveolar dead space (VDalv), specifically techniques that use direct measurement, so as to provide a gold standard that is mainly or only limited by precision in measurement.

A glossary of the main terms/abbreviations used in this disclosure is given below:

FACO₂—Fractional alveolar partial pressure of CO₂; may be computed as PACO₂/P_{B}

FaCO₂—Fractional arterial partial pressure of CO₂

f_{B}—Breathing frequency

FCO₂—Fractional partial pressure of CO₂; the partial pressure of CO₂/P_{B}

FĒCO₂—Fractional mixed expired partial pressure of CO₂; PĒCO₂/P_{B}

FICO₂—Fractional concentration of inspired CO₂

FRC—Functional residual capacity; that volume of gas in the lung at the end of a normal exhalation. The partial pressures of O₂ and CO₂ in the FRC are equal to those in the capillary blood

PACO₂—Alveolar partial pressure of CO₂; the partial pressure of CO₂ in alveoli that has equilibrated with the partial pressure of CO₂ of the blood in the alveolar capillaries

PaCO₂—Partial pressure of CO₂ in arterial blood

P_{B}—Barometric pressure

PCO₂—Partial pressure of CO₂

PĒCO₂—Mixed expired partial pressure of CO₂; for example, the partial pressure of CO₂ in a bag containing several exhaled breaths; the net partial pressure of CO₂ after mixing alveolar gas and dead-space gas

P_{ET}CO₂—Partial pressure of carbon dioxide in exhaled gas at end expiration

P_{ET}O₂—Partial pressure of oxygen in exhaled gas at end expiration

Pv̅CO₂—Mixed venous partial pressure of CO₂ which is the PCO₂ in the blood returning from various parts of the body, mixed together; this is the PCO₂ of the blood entering the alveoli.

\dot{Q}—Cardiac output; perfusion (L/min)

\dot{Q}s—Shunt flow; that part of pulmonary blood flow that does not undergo gas exchange before becoming part of the arterial blood (L/min)

SGD—sequential gas delivery; inspiratory gas is divided into a volume of first gas (G1), which is intended to enter the alveoli, followed by a second gas (G2), the volume of which is at least that of the anatomical dead space (VDanat). The composition of second gas (G2) may be equivalent to gas that equilibrated with capillary blood in alveoli on the previous breath.

V_{A}—Volume of gas entering the alveoli

\dot{V}_{A}—Alveolar ventilation; volume of gas per minute in the alveoli, whether perfused or not. The latter is the ventilation of the alveolar dead space (VDalv)

\dot{V}_{E}—Minute ventilation; total volume of gas breathed per minute

VDphys—Physiological dead space; the part of the tidal volume V_{T} that does not undergo gas exchange; it is composed of anatomical dead space (VDanat) and alveolar dead space (VDalv)

VDalv—Alveolar dead space; the volume of gas that enters alveoli but does not undergo gas exchange. This is because these alveoli are not perfused. This part of the lung has a high ratio of ventilation to perfusion, i.e., a high \dot{V}/\dot{Q}. In reality, there is some ventilation and some perfusion in most alveoli. If the gas reaches a region where some gas exchange takes place, that gas is functionally divided into the equivalent of a volume that has undergone full gas exchange (matched \dot{V}/\dot{Q}) and a volume that has undergone no gas exchange (high \dot{V}_{E}/\dot{Q}, i.e., VDalv).

VDanat—Anatomical dead space; the volume of the lung incapable of gas exchange such as trachea, major bronchi, and branch bronchi down to alveolar ducts leading to the alveoli; the VDanat is about 2 ml/kg weight (see FIG. 1)

V_{T}—Tidal volume; the volume of inspired gas (mL)

VTalv—Alveolar tidal volume; the volume of gas distributed each breath to the alveoli, whether or not involved in gas exchange

NOTE: Fractional concentration is equal to the partial pressure of the gas divided by the barometric pressure (P_{B}), e.g., for CO₂, F_{ET}CO₂=P_{ET}CO₂/P_{B}. When a fractional concentration of CO₂ is multiplied by a gas volume, it results in a volume of CO₂. As barometric pressure is readily measurable, fractional concentration and partial pressure are used interchangeably herein.

NOTE: In general, upper case “A” indicates alveolar and lower case “a” indicates arterial.

The matching of ventilation and perfusion (\dot{V}/\dot{Q}) in the alveoli is crucial for providing adequate gas exchange and optimizing O₂ uptake and CO₂ elimination, as well as regulating the optimal partial pressures of each in the arterial blood. An adequate partial pressure of O₂ in arterial blood (PaO₂) is required to provide a partial pressure gradient for diffusion of O₂ into the tissues. An optimal partial pressure of CO₂ (PCO₂) is required to maintain pH in the blood and intracellular fluid for enzymes to work at optimal efficiency. The latter is controlled by feedback loops between blood partial pressure of CO₂ (PaCO₂) as sensed by the carotid bodies, and tissue partial pressure of CO₂ (PCO₂) in the medulla of the brain and the consequent adjustment of minute ventilation (\dot{V}_{E}).

In the perfused alveoli, the partial pressures of CO₂ and O₂ (PCO₂ and PO₂) equilibrate with the blood, resulting PCO₂ equal to that in the arterial blood (PaCO₂), say, about 40 mmHg The PCO₂ in the alveoli is termed alveolar PCO₂ and designated PACO₂.

If there were no blood that crossed from mixed venous side to the arterial side without undergoing gas exchange (i.e., shunt flow Qs), the arterial and alveolar partial pressures of CO₂ (PaCO₂ and PACO₂) would be equal. To the extent that there is Qs, PaCO₂ may exceed PACO₂. As Qs is almost always small, and the CO₂ content in the mixed venous blood is very close to that in the arterial blood, despite the difference in PCO₂, except for very large shunt flow, there is very little difference between PaCO₂ and PACO₂.

If blood flow is reduced to a part of the lung, for example by blockage of branches of the pulmonary artery such as by blood clot embolization (e.g., blocking off a major blood vessel to a part of the lung), the ventilation to that part of the lung is wasted (i.e., that part of the lung has high \dot{V}/\dot{Q}). The blood that would have perfused that lung is redistributed to other parts of the lung where ventilation is intact. In these alveoli, where perfusion and ventilation are intact, the alveolar gas equilibrates with the capillary blood such that the alveolar partial pressure of CO₂ (PACO₂) is normal, as then is the arterial partial pressure of CO₂ (PaCO₂).

In the alveoli that are not perfused, PCO₂ remains a function of the gas that was left in that space at the end of exhalation, plus what was inhaled. The latter consists of an initial volume of equilibrated gas left in the anatomical dead space (VDanat) from the previous breath, and room air with a partial pressure of CO₂ of 0 mmHg. At end inhalation, these gases mix, but no equilibration takes place. On exhalation, the end tidal partial pressure of CO₂ (P_{ET}CO₂) consists of a mixture of alveolar gas from perfused alveoli (PCO₂ is about 40 mmHg) and gas from the alveolar dead space (VDalv), PCO₂ of about 0 mmHg, mixed together. The greater the contribution from the alveolar dead space (VDalv), the lower the P_{ET}CO₂, and the less the measured P_{ET}CO₂ reflects the actual PACO₂ or PaCO₂.

Discussed herein are techniques to directly measure the fractional alveolar partial pressure of CO₂ (FACO₂) by reducing or eliminating the dilution effect of the alveolar dead space (VDalv) on the end tidal partial pressure of CO₂ (P_{ET}CO₂), thus making the end tidal partial pressure of CO₂ (P_{ET}CO₂) about equal to FACO₂. The present techniques apply this determined FACO₂ proxy, as in Equation 1 below, to calculate the ratio of the alveolar dead space to tidal volume (VDalv/V_{T}) and, further, when tidal volume (V_{T}) is measured or otherwise known, to calculate the alveolar dead space (VDalv).

Considering the alveolar partial pressure of CO₂ (PACO₂), which is taken to be equal to the arterial partial pressure of CO₂ (PaCO₂) as discussed above, then the amount of alveolar dead space (VDalv) that would be required to produce the measured end tidal partial pressure of CO₂ (P_{ET}CO₂) can be computed as:

(V_{T}−VDanat−VDalv)×FACO₂=VT×FĒCO₂

or by dividing both sides by VT and FACO₂ and simplifying:

1−Vdalv/(V_{T}−VDanat)=FĒCO₂/FACO₂

VDalv/(V_{T}−VDanat)=1−FĒCO₂/FACO₂ (Eq. 1),

with the assumptions that all of the CO₂ exhaled comes from the perfused alveoli and that the alveolar dead space (VDalv) contributes no CO₂.

As a simplification, this can be estimated for single breaths where the fractional end tidal partial pressure of CO₂ (F_{ET}CO₂) is determined only by the fractional alveolar partial pressure of CO₂ (FACO₂) and the alveolar dead space (VDalv). Use of sequential gas delivery reduces or eliminates the anatomical dead space (VDanat) from the calculation, so that looking only at the volume of gas entering the alveoli (V_{A}) where:

V_{T}−VDalv=V_{A}=G1/f_{B}

and allowing the substitution of F_{ET}CO₂ for FĒCO₂, thus it can be said that:

(V_{T}−VDalv)×FACO₂=V_{T}×F_{ET}CO₂

1−VDalv/(V_{T}−VDanat)=F_{ET}CO₂/FACO₂

VDalv/V_{A}=1−F_{ET}CO₂/FACO₂ (Eq. 2).

In the above, F_{ET}CO₂ and V_{T} may be measured, leaving three unknowns: VDanat, FACO₂, and VDalv. VDanat may be eliminated with sequential gas delivery, as will be discussed below. FACO₂ and VDalv are related to each other and represent only one degree of freedom. Hence, knowing one determines the other. PACO₂ can be estimated from the Phase III (see above and FIGS. 1A and 1B) or from the PaCO₂. The techniques discussed herein provide for measuring F_{ET}CO₂ and PACO₂ and thus, via Equation 2, determining VDalv using direct measurement.

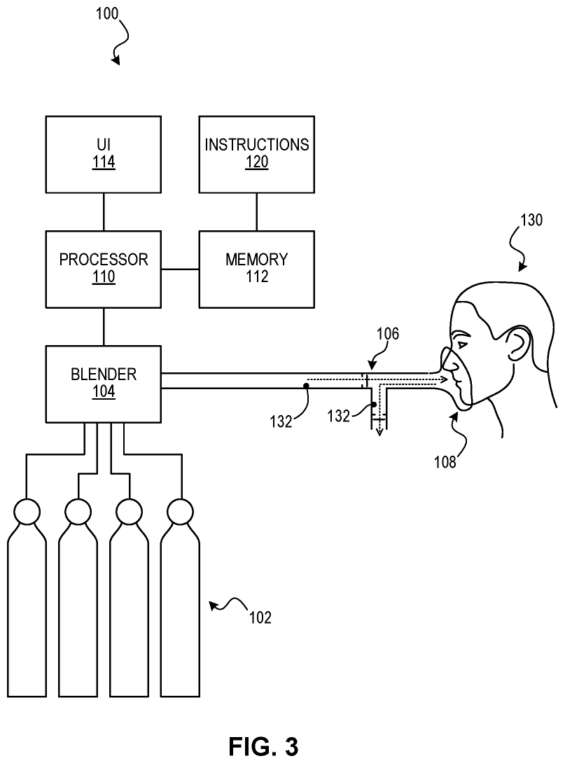

FIG. 3 shows a device 100 to provide sequential gas delivery to a subject 130 and determine the subject's alveolar dead space (VDalv). The device 100 includes gas supplies 102, a gas blender 104, a mask 108, a processor 110, memory 112, and a user interface device 114. The device 100 may be configured to control end-tidal PCO₂ and end-tidal PO₂ by generating predictions of gas flows to actuate target end-tidal values. The device 100 may be an RespirAct™ device, made by Thornhill Medical™ of Toronto, Canada, specifically configured to implement the techniques discussed herein. For further information regarding sequential gas delivery, U.S. Pat. No. 8,844,528, US Pub. 2018/0043117, and U.S. Pat. No. 10,850,052, which are incorporated herein by reference, may be consulted.

The gas supplies 102 may provide carbon dioxide, oxygen, nitrogen, and air, for example, at controllable rates, as defined by the processor 110. The following gas mixtures may be used. Gas A: 10% O₂, 90% N₂; Gas B: 10% O₂, 90% CO₂; Gas C: 100% O₂; and a calibration gas: 10% O₂, 9% CO₂, 81% N₂.

The gas blender 104 is connected to the gas supplies 102, receives gasses from the gas supplies 102, and blends received gasses as controlled by the processor 110 to obtain a gas mixture, such as a first gas (G1) and a second gas (G2) for sequential gas delivery.

The second gas (G2) is a neutral gas in the sense that it has about the same PCO₂ as the gas exhaled by the subject 130, which includes about 4% to 5% CO₂. In some examples, the second gas (G2) may include gas actually exhaled by the subject 130. The first gas (G1) has a composition that meets the respiratory needs of the subject and has a lower PCO₂ than that of the second gas (G2). For example, the first gas (G1) may be air (which typically has about 0.04% CO₂), may consist of 21% oxygen and 79% nitrogen, or may be a gas of similar composition, preferably without any appreciable CO₂.

The processor 110 may control the blender 104, such as by electronic valves, to deliver the gas mixture in a controlled manner.

The mask 108 is connected to the gas blender 104 and delivers gas to the subject 130. A valve arrangement 106 may be provided to the device 100 to limit the subject's inhalation to gas provided by the blender 104 and limit exhalation to the room. An example valve arrangement 106 includes an inspiratory one-way valve from the blender 104 to the mask 108, a branch between the inspiratory one-way valve and the mask 108, and an expiratory one-way valve at the branch. Hence, the subject 130 inhales gas from the blender 104 and exhales gas to the room.

The gas supplies 102, gas blender 104, and mask 108 may be physically connectable by conduits, such as tubing, to convey gas. Any number of sensors 132 may be positioned at the gas blender 104, mask 108, and/or conduits to sense gas flow rate, pressure, temperature, and/or similar properties and provide this information to the processor 110. Gas properties may be sensed at any suitable location, so as to measure the properties of gas inhaled and/or exhaled by the subject 130.

The processor 110 may include a central processing unit (CPU), a microcontroller, a microprocessor, a processing core, a field-programmable gate array (FPGA), an application-specific integrated circuit (ASIC), or a similar device capable of executing instructions. The processor may be connected to and cooperate with the memory 112 that stores instructions and data.

The memory 112 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface device 114 may include a display device, touchscreen, keyboard, buttons, and/or similar to allow for operator input/output.

Instructions 120 may be provided to carry out the functionality and methods described herein. The instructions 120 may be directly executed, such as a binary file, and/or may include interpretable code, bytecode, source code, or similar instructions that may undergo additional processing to be executed.

The instruction 120 perform sequential gas delivery by controlling the device 100 to deliver a first volume of a first gas (G1) to the subject 130 over a first portion of an inspiration by the subject 130. The first volume is selected to be less than or equal to an estimated or expected alveolar volume (V_{A}) of the subject 130 when the subject is breathing normally. The first gas (G1) has a PCO₂ that is less than the PCO₂ in the gas exhaled by the subject 130 and preferably no significant amount of CO₂. The instructions 120 deliver a second volume of a second, neutral gas (G2) to the subject 130 over a second portion of the inspiration. The second gas is a neutral gas that has a PCO₂ corresponding to the PCO₂ in the exhaled gas and preferably the same amount of CO₂ as present in the previously exhaled breath. The second gas (G2) is unlimited in the sense that during normal or deep breathing, the end of the inspiration will contain as much second gas (G2) as needed.

The instructions 120 measure a PCO₂ at the end of an exhalation by the subject 130 occurring after delivery of the first and second gases (G1, G2), that is the P_{ET}CO₂, while the subject breathes.

The instructions 120 may measure the subject's P_{ET}CO₂ during spontaneous normal breathing and deep breathing over a plurality of respiratory cycles, so as to allow measurements to stabilize and to allow for a mean to be computed.

The instructions 120 apply Equation 2 or equivalent to compute the alveolar dead space (VDalv) using the normal breathing P_{ET}CO₂ and the subject's PaCO₂ as the PACO₂.

Instead of invasively measuring the subject's PaCO₂, the subject's PaCO₂ or PACO₂ can be measured by having the subject breathe deeply, with the instructions 120 measuring P_{ET}CO₂ at exhalation when the subject is breathing deeply. The deep breathing P_{ET}CO₂, as will be explained below, may then be taken as the PaCO₂ or PACO₂ (or the fractional equivalents). This is possible because, in the dead space alveoli, the larger the breath the closer the PCO₂ approaches the equilibrated value. Hence, with each deep breath, the residual PCO₂ in the dead space alveoli approach the equilibrated value. As the dead space PCO₂ approaches the equilibrated value, the P_{ET}CO₂ approaches the equilibrated value, that is, the PACO₂. The instructions 120 then take the deep breathing P_{ET}CO₂ to be the PaCO₂ or, more specifically, the PACO₂ term in Equation 2.

Hence, the instructions 120 facilitate the computation of subject's alveolar dead space (VDalv) based on a ratio of P_{ET}CO₂ during normal breathing to P_{ET}CO₂ during deep breathing. The effect of VDalv on F_{ET}CO₂ may be reduced or eliminated, so as to make F_{ET}CO₂ equal to FACO₂.

The instructions 120 may further output the determined alveolar dead space (VDalv), or a representation thereof, at the user interface device 114. Further, the instructions 120 may cause the user interface device 114 to prompt the operator of the device 100 to operate the device 100 and instruct the subject 130, as discussed herein.

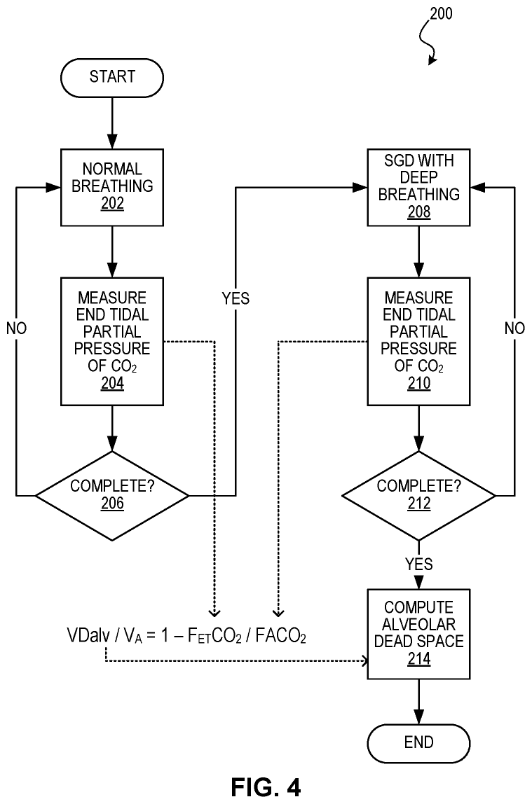

FIG. 4A shows an example method 200 of determining alveolar dead space. The method 200 may be implemented by instructions 120.

At block 202, the subject breathes normally. Sequential gas delivery may be used, as discussed above. The first gas (G1) is delivered at a volume less than or equal to an estimated or expected alveolar volume V_{A} of the subject. The neutral gas (G2), if not rebreathed gas, may have its PCO₂ adjusted continually to match exhaled gas. During breathing, at block 204, the subject's P_{ET}CO₂ is measured. This continues for a time, via block 206, such as 20 seconds, 30 seconds, 1 minute, 2 minutes, or longer.

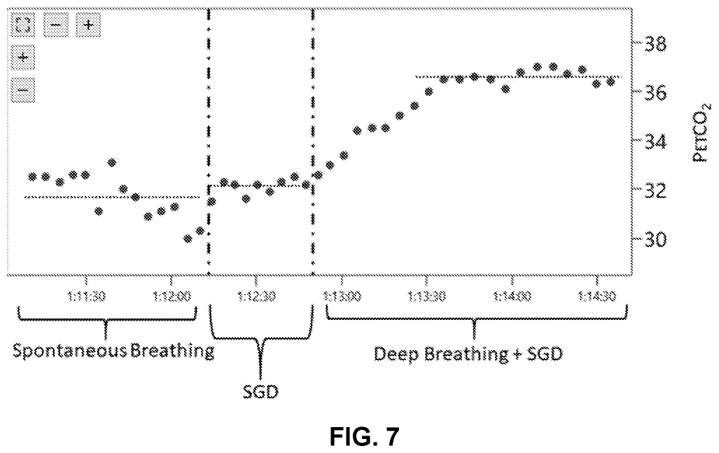

At block 208, the subject breathes deeply with sequential gas delivery. The first gas (G1) is delivered at the same volume in block 202. During deep breathing, at block 210, the subject's P_{ET}CO₂ is measured. This continues for a time, via block 212, such as 20 seconds, 30 seconds, 1 minute, 2 minutes, or longer. The time may be governed by the slope or change in the curve of the measured P_{ET}CO₂ (see FIG. 7), such that blocks 208 and 210 are performed until the P_{ET}CO₂ stops its increasing trend and levels out.

At block 214, the alveolar dead space (VDalv) is computed using the ratio of the P_{ET}CO₂ measured from blocks 204 and 210, that is, by applying Equation 2. VDalv may be outputted as a ratio to V_{A} or as an absolute value provided that V_{A} is determined.

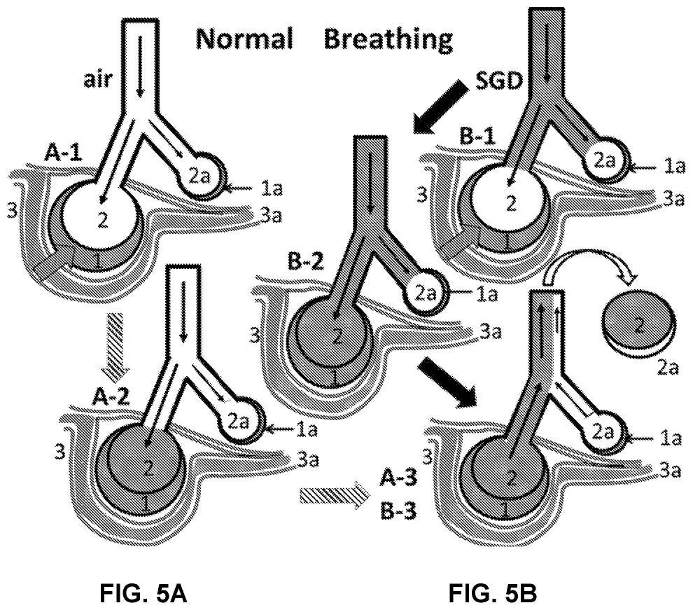

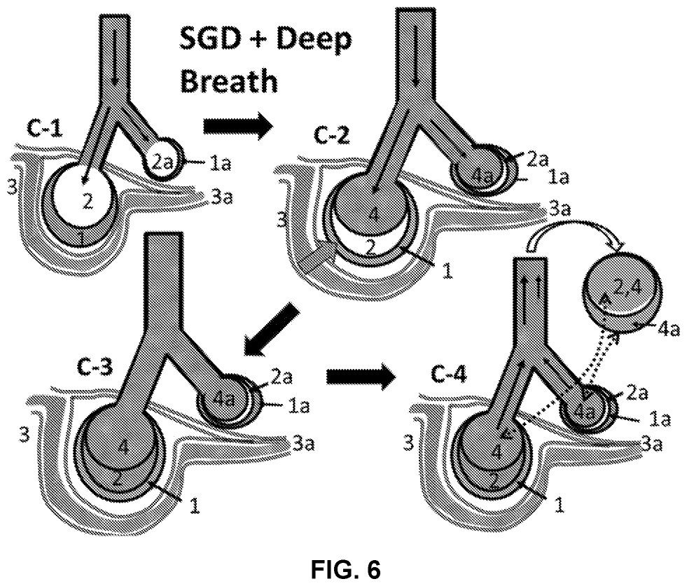

Further explanation of the above is provided with references to FIGS. 5A, 5B, and 6, which show a two-region alveolar model with one alveolar region perfused (at left) and one not (at right). In these figures, the functional residual capacity is show at 1, the functional residual capacity in dead space alveoli plus the equilibrated gas from the previous breath inhaled from the anatomical dead space is show at 1a, the part of V_{T} entering the alveolus is shown at 2, mixed venous blood with Pv̅CO₂ is shown at 3, and PCO₂ in blood equilibrated with alveolar gas 1, 2 is shown at 3a.

With reference to FIG. 5A, during normal/spontaneous breathing without sequential gas delivery, inspiration occurs at A-1 to draw in fresh gas 2, 2a into the respective perfused alveolar region and non-perfused alveolar region, which respectively contain residual gas 1, 1a. Equilibration of CO₂ and O₂ between the gas in the alveoli and the pulmonary capillaries, at the perfused alveolar region, occurs at A-2. There is no equilibration in alveolar dead space. Exhalation at A-3 provides a mix of equilibrated gas 1, 2 and dead space gas 1a, 2a.

With reference to FIG. 5B, during sequential gas delivery with normal/spontaneous breathing, during inspiration at B-1 a first gas (G1) is distributed to both perfused and non-perfused (dead space) alveoli (2, 2a) and the latter part of inspiration includes second gas (G2) with PCO₂ equal to equilibrated gas. The second gas (G2) fills the anatomical dead space. It does not matter if the second gas (G2) enters the gas exchange alveoli (see FIG. 6), so it is depicted as ending in the anatomical dead space. During gas exchange at B-2, the fresh gas or air 2 inhaled into the alveoli equilibrate with blood in alveolar capillaries. At exhalation B-3, this equilibrated gas mixes with alveolar dead space gas and the resulting P_{ET}CO₂ is substantially the same as normal breathing (see also FIG. 7).

With sequential gas delivery at spontaneous respiration and normal V_{T}, V_{T} is divided into a first gas (G1) which provides gas to all the alveoli (V_{A}) followed by a second gas (G2) which has PCO₂ and optionally PO₂ equal to equilibrated gas. G2 fills only the VDanat, as shown in FIG. 5B at B-1. If all of the alveoli are capable of gas exchange, G1 is equal to the alveolar ventilation (\dot{V}_{A}) as long as V_{T}≥V_{A}+VDanat. To the extent that there is VDalv in the V_{A}, during exhalation, the proportion of gas from perfused alveoli and VDalv is the same as with normal breathing, as shown in FIG. 5B at A-3 and B-3, so that P_{ET}CO₂ is also the same, as shown in FIG. 7.

With reference to FIG. 6, during sequential gas delivery with deep breathing, inspiration at C-1 includes inspiration of first gas (G1). The alveolar gas enters the perfused alveoli 2 and the dead space alveoli 2a. The subject takes a bigger breath at C-2 and draws into the perfused alveoli 4 and non-perfused alveoli 4a more equilibrated gas (G2). In the perfused alveoli, the inhaled fresh gas equilibrates at C-3 with the mixed venous gas. Because the volume of fresh gas is the same as at rest or regular/spontaneous (non-deep) breathing, the equilibrium PCO₂ is the same as at rest, i.e., PACO₂. In the dead space alveoli, the inhaled fresh gas (G1) is diluted by equilibrated gas (G2), and PCO₂ rises towards that in equilibrated gas (G2). Regardless of the size of the breath, the perfused alveolar PCO₂ is unchanged. However, in the dead space alveoli, the bigger the breath, the closer the PCO₂ approaches the equilibrated value. With each breath, the residual PCO₂ in the dead space alveoli approach the equilibrated value. As the dead space PCO₂ approaches the equilibrated value at C-4, the P_{ET}CO₂ approaches the equilibrated value, that is, PACO₂.

In other words, the subject takes a deep breath at C-2, and the V_{A}, both the part with perfused alveoli and that with dead space alveoli, now contains its FRC+G1+an expanded volume of G2. This gas enters the perfused alveoli 4 at C-2 and the dead space 4a. At equilibration at C-3, the perfused alveoli equilibrate to PACO₂ as in normal breathing, but the PCO₂ in the VDalv now rises from about 0 towards P_{ET}CO₂. Thus, on exhalation, P_{ET}CO₂ approaches PACO₂.

Deep breaths with sequential gas delivery send equilibrated gas to the VDalv and dilute out first gas (G1), and wash out the previous gas, such that the PCO₂ in the VDalv approaches the PACO₂. Thus, P_{ET}CO₂ approaches PACO₂. This PACO₂ is used in Equation 2 to calculate VDalv/V_{A}.

FIG. 7 shows a VDalv/V_{A} test plotted as P_{ET}CO₂ vs. time. At left, initial normal/free breathing occurs with breath-to-breath variation. This is followed by sequential gas delivery at a normal breathing rate with less variation, which is followed by sequential gas delivery with deep breaths. Each dot is a P_{ET}CO₂ measurement. It took about 30 seconds to wash out the VDalv and come to a new equilibrium P_{ET}CO₂.

The techniques described above may be used to assess a medical condition such as heart failure, congestive heart failure, adult respiratory distress syndrome, lung function after lung transplant, lung function after transplant, sepsis, trauma, or pulmonary and systemic disease. That is, a directly measurable alveolar dead space may be useful is assessing such conditions. Moreover, measuring alveolar dead space may be used as a marker for the severity and duration of congestive heart failure.

### EXAMPLE

An example of measuring VDalv using the techniques described herein will now be discussed.

1. A subject is in a resting steady state of breathing and oxygen consumption;

2. The subject breathes via a tight-fitting mask or mouthpiece, endotracheal tube, or laryngeal mask such that:
a. The volume and concentrations of CO₂ in all inspired gases are controlled and measured,
b. PCO₂ is measured continuously on exhalation,
c. P_{ET}CO₂ is measured breath-by-breath, and
d. Tidal volumes are measured breath-by-breath;

3. VDanat is calculated as 2.2 ml/kg body weight+1 ml×age or with a similar method;

4. Sequential gas delivery (SGD) is performed by:
a. Measuring the breathing frequency (f_{B}) and tidal volumes (V_{T}) in a steady state,
b. Calculating VDalv by one of the following:
   i. Calculate the approximate anatomical dead space (VDanat) from tables,
   ii. Assume VDanat to be 2.2 ml/kg+1 ml×age,
   iii. Measure the VDanat by progressive reduction of the first gas (G1) below VE and observe inflection to increase in P_{ET}CO₂. VDanat=difference in flow between \dot{V}_{E} and at flow at the upward inflection, divided by breathing frequency (f_{B}),
   iv. Set first gas (G1) flow is equal to V_{T}−VDanat×f_{B},
   v. Set PCO₂ of second gas (G2) to P_{ET}CO₂ of previous breath,

5. During SGD there is no change in P_{ET}CO₂ from free breathing to SGD (see FIG. 7);

6. Subject begins to hyperventilate taking deep breaths. The time constant of washout of VDalv will be FRC/(V_{T}×f_{B}). For example, assuming FRC of 4 L, breaths of 2 L, breathing frequency of 20/min, and a time constant of washout of the lung of 3 seconds, therefore at 9 second there will be 95% washout of VDalv with the second gas (G2); and

7. Measure P_{ET}CO₂ during deep breathing and take the P_{ET}CO₂ as PACO₂. To the extent that the PCO₂ in the alveolar dead space approaches the P_{ET}CO₂, the P_{ET}CO₂ will rise and asymptote at the PACO₂, as shown in FIG. 7; and

8. Compute VDalv using Equation 2.

In view of the above, it should be apparent that a convenient, quick, non-invasive, and accurate measurement of alveolar dead space (VDalv) may be realized using the techniques discussed herein. Further, since all inputs (namely V_{T}, F_{ET}CO₂ during normal breathing, and FACO₂ as F_{ET}CO₂ during deep breathing) to compute VDalv are measurable or directly computable from measurements, the present disclosure provides a gold standard measure that depends only on the precision of the measurements and not on approximations or unfounded assumptions.

It should be recognized that features and aspects of the various examples provided above can be combined into further examples that also fall within the scope of the present disclosure. In addition, the figures are not to scale and may have size and shape exaggerated for illustrative purposes.

We claim:

1. A method of determining alveolar dead space of a subject, the method comprising:
measuring an end tidal partial pressure of carbon dioxide during a sequence of normal breaths of the subject;
during a sequence of deep breaths by the subject, delivering a first volume of a first gas to the subject over a first portion of each inspiration by the subject, the first volume being less than or equal to an expected alveolar volume of the subject when the subject is breathing normally, and delivering a second volume of a second gas to the subject over a second portion of each inspiration, wherein the second gas comprises a neutral gas;
measuring an end tidal partial pressure of carbon dioxide during the sequence of deep breaths; and
computing the alveolar dead space using the end tidal partial pressure of carbon dioxide measured during the sequence of normal breaths and the end tidal partial pressure of carbon dioxide measured during the sequence of deep breaths.

2. The method of claim 1, comprising computing the alveolar dead space based on a ratio of the end tidal partial pressure of carbon dioxide measured during the sequence of normal breaths to an alveolar partial pressure of carbon dioxide of the subject, wherein the alveolar partial pressure of carbon dioxide of the subject is considered to be the end tidal partial pressure of carbon dioxide measured during the sequence of deep breaths.

3. The method of claim 2 wherein:
the first gas has a partial pressure of carbon dioxide that is less than a partial pressure of carbon dioxide of a gas exhaled by the subject; and
the second gas has a partial pressure of carbon dioxide corresponding to the partial pressure of carbon dioxide of the gas exhaled by the subject.

4. The method of claim 3, wherein the first volume is less than or equal to an expected alveolar volume of the subject when the subject is breathing normally.

5. Use of the method of claim 1 in an assessment of a medical condition selected from a group consisting of heart failure, congestive heart failure, adult respiratory distress syndrome, lung function after lung transplant, lung function after transplant, sepsis, trauma, and pulmonary and systemic disease.

6. A device for determining alveolar dead space of a subject, the device comprising:
a user interface device;
a gas blender to receive source gases; and
a processor connected to the user interface device and the gas blender to control the supply of gas to the subject, wherein the processor is to:
control the gas blender to deliver a first gas and a second gas sequentially to the subject during a period of deep breathing by the subject, wherein a volume of the first gas is about an expected alveolar volume of the subject when the subject is breathing normally, wherein the second gas is a neutral gas, and wherein the first gas has a partial pressure of carbon dioxide that is less than that of the second gas;
measure an end tidal partial pressure of carbon dioxide of the subject during the period of deep breathing;
compute a representation of alveolar dead space using the end tidal partial pressure of carbon dioxide measured during deep breathing and an end tidal partial pressure of carbon dioxide of the subject obtained during normal breathing; and
output the indication of the alveolar dead space at the user interface.

7. The device of claim 6, wherein the processor is further to:
deliver the first gas and the second gas sequentially to the subject during a period of normal breathing; and
measure the end tidal partial pressure of carbon dioxide of the subject during the period of normal breathing.

8. The device of claim 6, wherein the processor is to compute the representation of alveolar dead space based on a ratio of the end tidal partial pressure of carbon dioxide of the subject obtained during normal breathing to an alveolar partial pressure of carbon dioxide of the subject, wherein the alveolar partial pressure of carbon dioxide of the subject is considered to be the end tidal partial pressure of carbon dioxide measured during deep breathing.

9. The device of claim 6, wherein the processor is to compute the representation of alveolar dead space as a ratio of the alveolar dead space to a volume of gas entering the alveoli.

10. Use of the device of claim 6 in an assessment of a medical condition selected from a group consisting of heart failure, congestive heart failure, adult respiratory distress syndrome, lung function after lung transplant, lung function after transplant, sepsis, trauma, and pulmonary and systemic disease.
